(19) United States
(12) Patent Application Publication
(10) Pub. No.: US 2010/0179854 A1
(43) Pub. Date: Jul. 15, 2010

(54) Scheduling System and Method

(76) Inventors: Steven L. Shafer, Closter, NJ (US); Thomas J. Shafer, Mountain View, CA (US); Nathan Esquenazi, Irvine, CA (US)

Correspondence Address: THOMPSON HINE L.L.P.;Intellectual Property Group, P.O. BOX 8801, DAYTON, OH, 45401-8801, US

(21) Appl. No.: 12/687,562

(22) Filed: Jan. 14, 2010

Related U.S. Application Data
(60) Provisional application No. 61/205,292, filed on Jan. 15, 2009.
(60) Provisional application No. 61/205,437, filed on Jan. 15, 2009.
(60) Provisional application No. 61/205,314, filed on Jan. 15, 2009.

(51) Int. Cl. G06Q 10/00; G06Q 50/00; A61B 5/00; G06F 15/16

(52) U.S. Cl. 705/9; 600/301; 709/206; 709/204

(57) ABSTRACT

A system for scheduling an appointment between a client and a provider including a monitoring and validation component that includes a transaction database, a validation engine and at least one transaction monitoring rule, and an Internet-based scheduling component the populates the transaction database, wherein the validation engine is configured to apply the transaction monitoring rules to the transaction database to identify the presence of exceptional transactions in the transaction database.

### PRIORITY

This application claims priority from U.S. Ser. No. 61/205,292 filed on Jan. 15, 2009, U.S. Ser. No. 61/205,437 filed on Jan. 15, 2009 and U.S. Ser. No. 61/205,314 filed on Jan. 15, 2009. The entire contents of all three of these provisional applications are incorporated herein by reference.

### FIELD

This application relates to scheduling systems and, more particularly, to Internet-based scheduling systems and, even more particularly, to systems and methods for monitoring and validating scheduling systems to maintain and assure system integrity.

### BACKGROUND

Appointments for services are commonly scheduled by telephone, wherein a client telephones the service provider to schedule the appointment. During the telephonic communication, the parties typically review their schedules, present available dates and times and, ideally, agree on an appointment time. This process can be time consuming and frustrating to both parties, particularly when it is difficult to identify a mutually acceptable date and time for the appointment.

In the medical profession, unforeseen complications and the unpredictability of preparation, procedure and recovery times make scheduling appointments particularly difficult. The difficulties are compounded by the typically need to match a patient (the client) not only with a specific doctor (the provided), but also with the required procedure room (e.g., an operating room or an examination room). In an effort to maximize value, medical service providers attempt to minimize idle time and maximize patient throughput. This can result in long, frustrating waits for patients that have arrived on time for their appointments.

Accordingly, those skilled in the art continue to seek new systems and method for scheduling appointments between clients and service providers.

### SUMMARY

In one aspect, the disclosed system for scheduling an appointment between a client and a provider may include a monitoring and validation component that includes a transaction database, a validation engine and at least one transaction monitoring rule, and an Internet-based scheduling component the populates the transaction database, wherein the validation engine is configured to apply the transaction monitoring rules to the transaction database to identify the presence of exceptional transactions in the transaction database.

In another aspect, the disclosed method for scheduling an appointment between a client and a provider may include the steps of (1) establishing a transaction database and at least one transaction monitoring rule, (2) receiving a request from the client to schedule an appointment with the provider, (3) providing the client with at least one available appointment time for the appointment, (3 ) receiving a selected appointment time from the client and logging the selected appointment time in the transaction database, wherein the selected appointment time is chosen from the available appointment times, (4) communicating the selected appointment time to the provider, (5) determining whether the provider has accepted the selected appointment time, (6) when the provider has accepted the selected appointment time, communicating a confirmation of the selected appointment time to the client and logging the acceptance of the selected appointment time in the transaction database, and (7) applying the transaction monitoring rule to the transaction database to identify the presence of exceptional transactions in the transaction database.

In another aspect, the disclosed system for scheduling an appointment between a client and a provider may include a monitoring and validation component that includes a transaction database, a validation engine and at least one transaction monitoring rule, and an Internet-based scheduling component configured to receive a request from the client to schedule an appointment with the provider, provide the client with at least one available appointment time for the appointment, receive a selected appointment time from the client, the selected appointment time being chosen from the at least one available appointment time, log the selected appointment time in the transaction database, communicate the selected appointment time to the provider and, when the provider has accepted the selected appointment time, communicate a confirmation of the selected appointment time to the client and log the acceptance of the selected appointment time in the transaction database, wherein the validation engine is configured to apply the transaction monitoring rule to the transaction database to identify the presence of exceptional transactions in the transaction database.

In another aspect, the disclosed method for scheduling an appointment between a client and a provider may include the steps of (1) scheduling a date for the appointment, (2) tracking utilization of a room to be used for the appointment using an electronic monitoring device, (3) determining a time of the appointment based on the tracking step and (4) notifying the client of the time.

In another aspect, the disclosed system for scheduling an appointment between a client and a provider may include an Internet-based scheduling component configured to schedule a date for the appointment, a procedure tracking component configured to track utilization of the room to be used for the appointment using an electronic monitoring device, a schedule optimization component configured to determining a time of the appointment based on data collected by the procedure tracking component, and a notification component configured to notify the client of the time of the appointment.

In another aspect, the disclosed method for scheduling an appointment between a client and a provider may include the steps of (1) scheduling the appointment, (2) establishing a pre-appointment preparation instruction for the appointment, (3) determining when the pre-appointment preparation instruction should be sent to the client and, after the determining step, (4) communicating the pre-appointment preparation instruction to the client.

In yet another aspect, the disclosed system for scheduling an appointment between a client and a provider may include an Internet-based scheduling component configured to schedule the appointment, a preparation maintenance component configured to generate a pre-appointment preparation instruction for the appointment, a patient preparation component configured to determine when the pre-appointment preparation instruction should be sent to the client, and a patient reminder component configured to communicate the pre-appointment preparation instruction to the client.

Other aspects of the disclosed scheduling system will become apparent from the following description, the accompanying drawings and the appended claims.

### BRIEF DESCRIPTION OF THE DRAWINGS

FIG. 1 is a block diagram of a first aspect of the disclosed scheduling system, which includes an Internet-based scheduling component and a monitoring and validation component;

FIG. 2 is a flow chart that illustrates the creation of the transaction monitoring rules of the monitoring and validation component of the scheduling system of FIG. 1;

FIG. 3 is a flow chart that illustrates the creation of the emergency contact database of the monitoring and validation component of the scheduling system of FIG. 1;

FIG. 4 is a flow chart that illustrates use of the Internet-based scheduling component of the scheduling system of FIG. 1;

FIG. 5 is a flow chart that illustrates the combination of the monitoring and validation component with the Internet-based scheduling component of FIG. 4;

FIG. 6 is a flow chart that illustrates how the validation engine of the monitoring and validation component of the scheduling system of FIG. 1 may identify exceptional transactions;

FIG. 7 is a flow chart that illustrates how the validation engine of the monitoring and validation component of the scheduling system of FIG. 1 may assess the integrity of the client (web) side of the scheduling system of FIG. 1;

FIG. 8 is a flow chart that illustrates web diagnostics of the validation engine of the monitoring and validation component of the scheduling system of FIG. 1;

FIG. 9 is a flow chart that illustrates how the validation engine of the monitoring and validation component of the scheduling system of FIG. 1 may assess the integrity of the provider (email) side of the scheduling system of FIG. 1;

FIG. 10 is a flow chart that illustrates email diagnostics of the validation engine of the monitoring and validation component of the scheduling system of FIG. 1;

FIG. 11 is a block diagram of a second aspect of the disclosed scheduling system, which includes an Internet-based scheduling component, a procedure tracking component, a schedule optimization component and a patient notification component;

FIG. 12 is a flow chart that illustrates the steps performed by the Internet-based scheduling component of the scheduling system of FIG. 11;

FIG. 13 is a flow chart that illustrates the steps performed by the procedure tracking component of the scheduling system of FIG. 11;

FIG. 14A and 14B are flow charts that illustrate the steps performed by the same-day schedule optimizing engine of the schedule optimization component of the scheduling system of FIG. 11;

FIG. 15 is a flow chart that illustrates the steps performed by the future schedule optimizing engine of the schedule optimization component of the scheduling system of FIG. 11;

FIG. 16 is a flow chart that illustrates the steps performed by the patient notification component of the scheduling system of FIG. 11;

FIG. 17 is a block diagram of an alternative embodiment of the second aspect of the disclosed scheduling system, which includes an intranet-based scheduling component, a procedure tracking component, a schedule optimization component and a patient notification component; and

FIG. 18 is a flow chart that illustrates the steps performed by the intranet-based scheduling component of the scheduling system of FIG. 17;

FIG. 19 is a block diagram of a third aspect of the disclosed scheduling system, which includes an Internet-based scheduling component, a preparation maintenance component, a patient preparation component and a patient reminder component;

FIG. 20 is a flow chart that illustrates the steps performed by the Internet-based scheduling component of the scheduling system of FIG. 19;

FIG. 21 is a flow chart that illustrates the steps performed by the preparation maintenance component of the scheduling system of FIG. 19;

FIG. 22 is a flow chart that illustrates the steps performed by the patient preparation component of the scheduling system of FIG. 19; and

FIG. 23 is a flow chart that illustrates the steps performed by the patient reminder component of the scheduling system of FIG. 19.

### DETAILED DESCRIPTION

In a first aspect, the disclosed scheduling system may provide clients with the ability to identify appointment times with a provider of services that are most convenient to the client, and to schedule those appointment times at any time, day or night. The disclosed scheduling system may identify available times in a provider's calendar, and may offer those available times to the client, wherein the client is free to select an appointment time that is most convenient for the client.

As used herein, “provider” broadly refers to any person, entity or thing that provides services during a scheduled appointment time, or any person, entity or thing working for or acting on behalf of a provider. For example, a provider may be a professional, such as a physician, a dentist, a psychologist, an attorney or an accountant. Other types of providers that may use the disclosed scheduling system will be left to the skilled artisan.

As used herein, “client” broadly refers to any consumer of services being provided by a provider. For example, a client may be a patient seeking the care of a medical doctor (i.e., the provider).

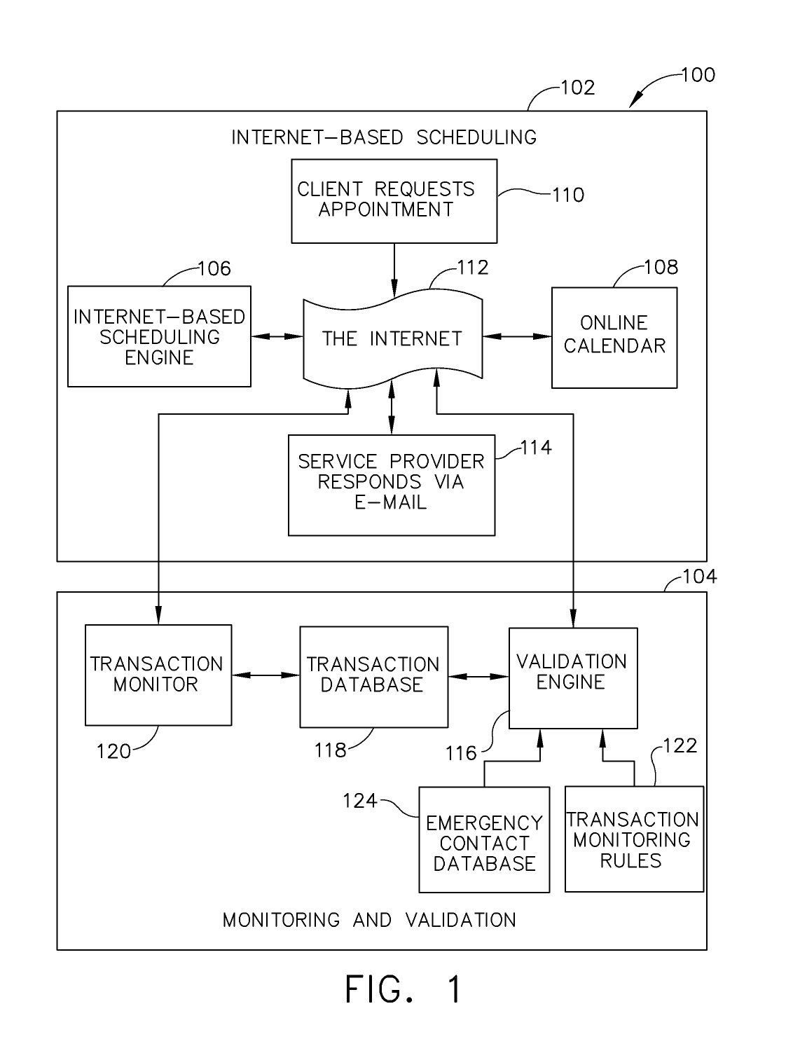

Referring to FIG. 1, one embodiment of the first aspect of the disclosed scheduling system, generally designated 100, may include an Internet-based scheduling component 102 and a monitoring and validation component 104. As will be discussed in greater detail below, the monitoring and validation component 104 may continuously monitor and validate the integrity of the scheduling system 100.

The Internet-based scheduling component 102 may include an Internet-based scheduling engine 106 and the provider's online calendar 108. As shown at block 110, a client may access the Internet-based Scheduling component 102 by way of the Internet 112 to request an appointment. Upon receiving a client's request for an appointment, the Internet-based scheduling engine 106 may access the online calendar 108 and may identify available appointment times (i.e., one or more specific dates and times when the provider is available for an appointment). The list of appointment times may then be presented to the client, and the client may select the most convenient time from the offered list of available dates and times. The client's selection may then be communication to the provider, at which time the provider may respond to the client, e.g., by email, as shown at block 114, and the appointment time may be scheduled in the online calendar 108.

The monitoring and validation component 104 of the scheduling system 100 may continuously assess the integrity of the Internet-based scheduling component 102. The monitoring and validation component 104 may include a validation engine 116, a transaction database 118 and a transaction monitor 120. The transaction monitor 120 may capture the requests for appointments from clients and the responses sent by the provider (block 114), and may store the collected data in the transaction database 118.

The validation engine 116 may regularly review the data in the transaction database 118, and may identify exceptional transactions based on transaction monitoring rules 122. The transaction monitoring rules 122 may be standard rules or may be customized rules established by the provider. The validation engine 116 may bring exceptional transactions to the attention of the provider.

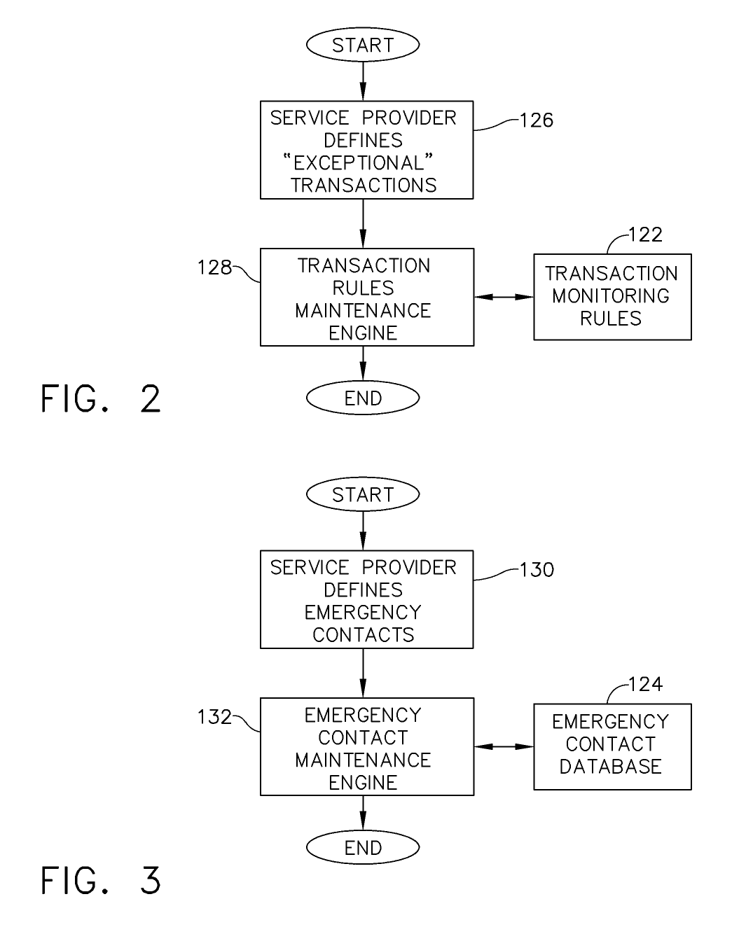

The transaction monitoring rules 122 may be created by the provider with the optional assistance of a transaction rules maintenance engine 128 (FIG. 2). For example, as shown in FIG. 2, the provider may define one or more “exceptional” transactions (block 126) and the transaction rules maintenance engine (block 128) may assist in the preparation of the rules to identify exceptional transactions. The generated rules may then be saved as the transaction monitoring rules 122.

As one example, an exceptional transaction may be a request for an appointment that is not answered within a given period of time (e.g., 24 hours). As another example, the service provider may create white lists of clients who are permitted to book appointments or black lists of clients who are not permitted to book appointments. Therefore, an exceptional transaction may be a transaction that violates the white list or black list rules. As another example, the service provider may define a maximum number of requests for an appointment from a particular client or IP address. Appointment requests that exceed this maximum may be flagged as exceptional. As yet another example, the service provider may specify a minimum number of transactions that will trigger an exception. For example, if no requests for appointments are received within 24 hours, the monitoring and validation component 104 may flag the lack of a client transaction as exceptional, and trigger web diagnostics. Similarly, if no responses are received from the provider within a given time (e.g., 24 hours), the monitoring and validation component 104 may flag the lack of a provider transaction as exceptional and trigger email diagnostics.

As a first specific example of an exceptional transaction, a client requests an appointment on a Monday afternoon, but by Wednesday afternoon there has been no response from the provider back to the client (block 114). The transaction monitoring rules 122 may be configured such that the validation engine 116 identifies the delay in responding to the client as exceeding a pre-determined threshold and reminds the provider to respond to the client.

As a second specific example of an exceptional transaction, a patient requests an appointment on Friday afternoon, but by Tuesday afternoon there has been no response from the provider back to the client (block 114). The transaction monitoring rules 122 may be configured such that the validation engine 116 identifies the delay in responding to the client as exceeding a pre-determined threshold and reminds the provider to respond to the client.

As a third specific example of an exceptional transaction, the system 100 sends a reminder to the provider, but does not receive a response (block 114) from the provider after two business hours. In such a situation, the validation engine 116 may activate diagnostics (e.g., sends an email to the provider, and then accesses the provider's email to determine that the IP address can be resolved, that the POP account responds properly to the login and password information, that the email generated by the system actually appears in the account Inbox, that the information in the email matches the email generated by the system). This information may be forwarded to the appropriate contact person using the emergency contact information in the emergency contact database 124 such that the contact person can assess whether the provider's email system is functioning and take the necessary steps to fix any problem.

As a fourth specific example of an exceptional transaction, every morning the system 100 may verify whether at least one client request has been received in the past 24 hours. If no request has been received in the past 24 hours, the validation engine 116 may initiate an appointment request on its own, and may verify that the request is properly processed. If the appointment request is not received, the validation engine 116 may undertake further server diagnostics (e.g., can the URL be resolved? Can the site be accessed? Is there an HTML problem with the site? Is the site working properly, but the information not being properly sent to the health care provider?). The validation engine 116 may contact the provider and the local Internet Service Provider, among others, and may advise them that the Internet site is apparently not accessible or not functioning properly, and may provide the information gleaned from the additional diagnostics.

As indicated above, if any portion of the system 100 appears to have failed, the validation engine 116 may initiate a series of diagnostics to identify the source of the failure. The validation engine 166 may also contact the provider or other appropriate contact person using the emergency contact information in the emergency contact database 124. The validation engine 116 may also initiate diagnostics on a regular basis, such as daily, to ensure system integrity.

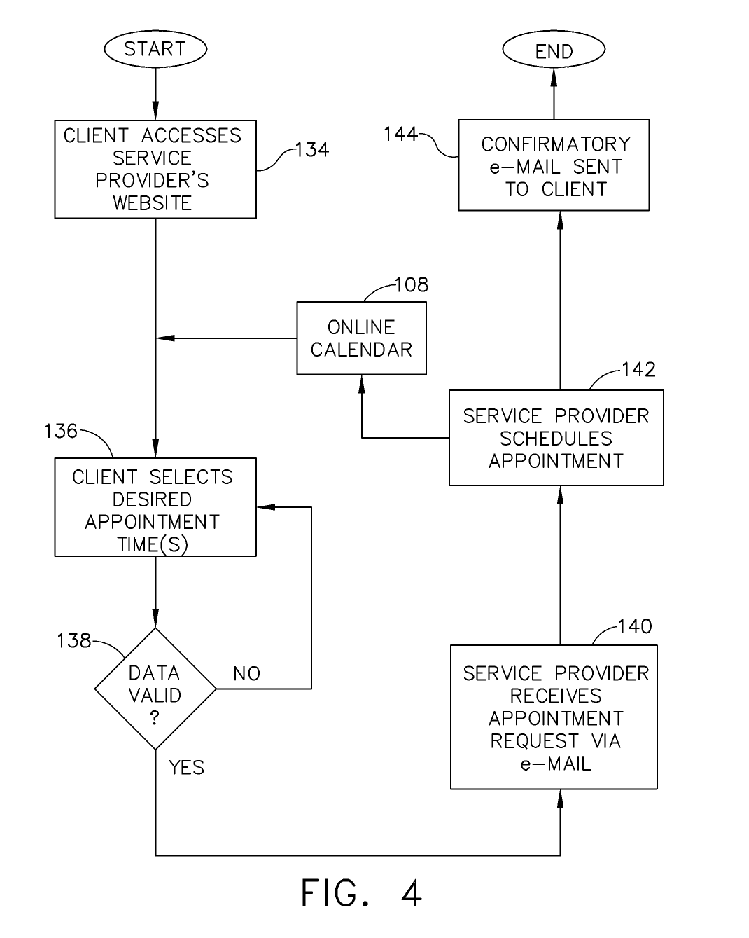

As shown in FIG. 3, the emergency contact database 124 may be created as follows. First, as shown at block 130, the provider may indentify a list of emergency contacts in the event that the monitoring and validation component 104 of the system 100 identifies a system failure. For example, the emergency contact database 124 may include contact information for the provider and IS personnel. Possible contact methods include phone, text message, email, fax or alphanumeric pager. The emergency contact maintenance engine 132 may capture this data, which may then be saved in the emergency contact database 124. Optionally, the emergency contact database 124 may also contain information on how to contact the administrator of the scheduling system 100, and the administration may also be notified in the event of a system failure.

Accordingly, as shown in FIG. 4, a client may request an appointment using the disclosed scheduling system 100. Specifically, at block 134, the client may access the provider's website and request an appointment. The Internet-based scheduling component 102 (FIG. 1) may access the provider's online calendar 108, may locate various available appointment times, and may present the available appointment times to the client. At block 136, the client may select one or more appointment times. For example, the client may be asked to select three acceptable time slots, which provides for alternatives in case several clients concurrently select the same slot. Once an appointment time has been selected, the system 100 may then validate the data, as shown at block 138. If the data is not valid, the client may again be asked to select an appointment. If the data is valid, the system may forward the request to the provider (block 140) and the provider may schedule the appointment (block 142). The scheduled appointment may be added to the online calendar 108 and a confirmatory message (e.g., email) may be sent to the client, as shown at block 144.

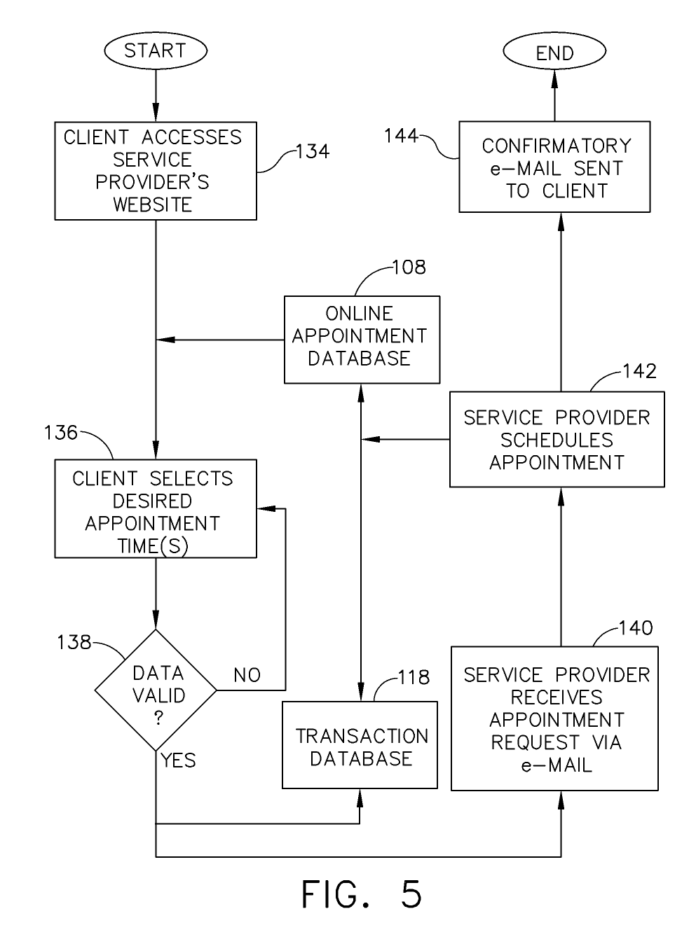

Referring now to FIG. 5, the monitoring and validation component 104 of the system 100 may build on the method shown in FIG. 4. Specifically, once the data are identified as valid (block 138), the transaction, including at least the date and time of the transaction, the client name and the date and time of the selected appointment, may be logged in the transaction database 118 concurrently with being forwarded to the provider (block 140). The transaction database 118 may also concurrently record when (1) the service provider schedules an appointment and (2) a response is forwarded to the client, among other things.

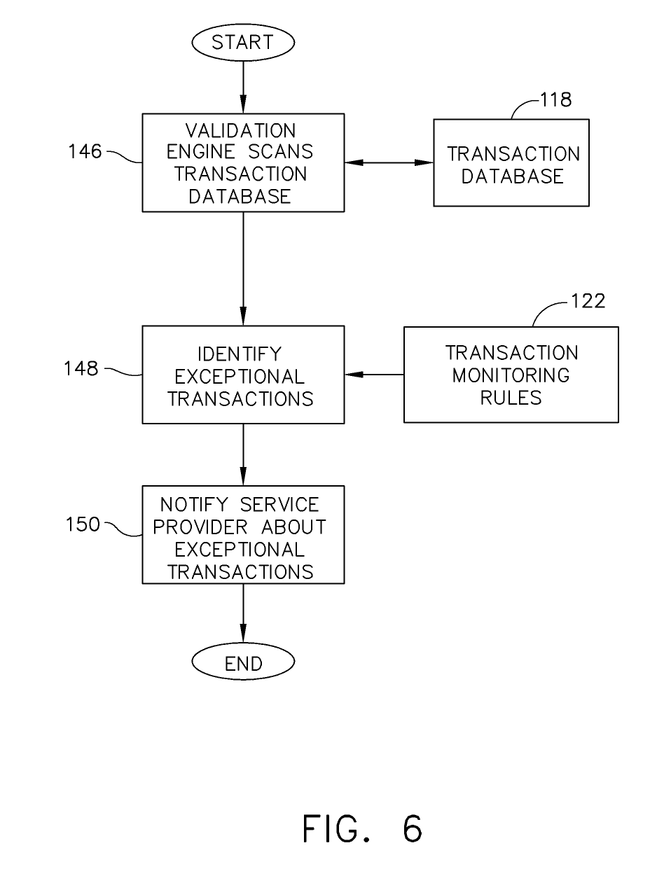

In one particular implementation, the validation engine 116 of the monitoring and validation component 104 of the scheduling system 100 may identify exceptional transactions as shown in FIG. 6. At block 146, the validation engine 116 (FIG. 1) may scan the transaction database 118 at regular intervals (e.g., every 6 hours). The transactions in the transaction database 118 may be screened against the transaction monitoring rules 122 to identify exceptional transactions, as shown at block 148. Then, at block 150, the provider may be sent a report describing newly identified exceptional transactions.

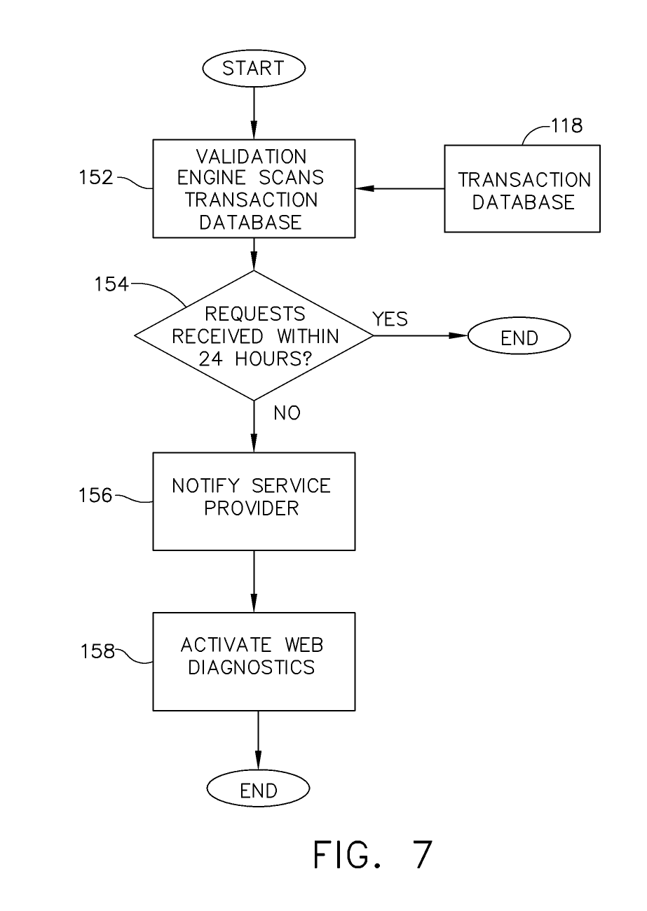

As noted above, the validation engine 116 (FIG. 1) may assess the integrity of the client (web) side of the Internet-based scheduling system 100. Referring to FIG. 7, in one particular implementation, beginning at block 152, the validation engine 116 may scan the transaction database 118 at regular intervals (e.g., every 10 minutes). Decision block 154 may query whether there have been any requests within a given time period (e.g., 24 hours). If not, then an exceptional transaction may be deemed to have occurred and a report may be sent to the provider (block 156) and the web diagnostics may be run by the validation engine (block 158).

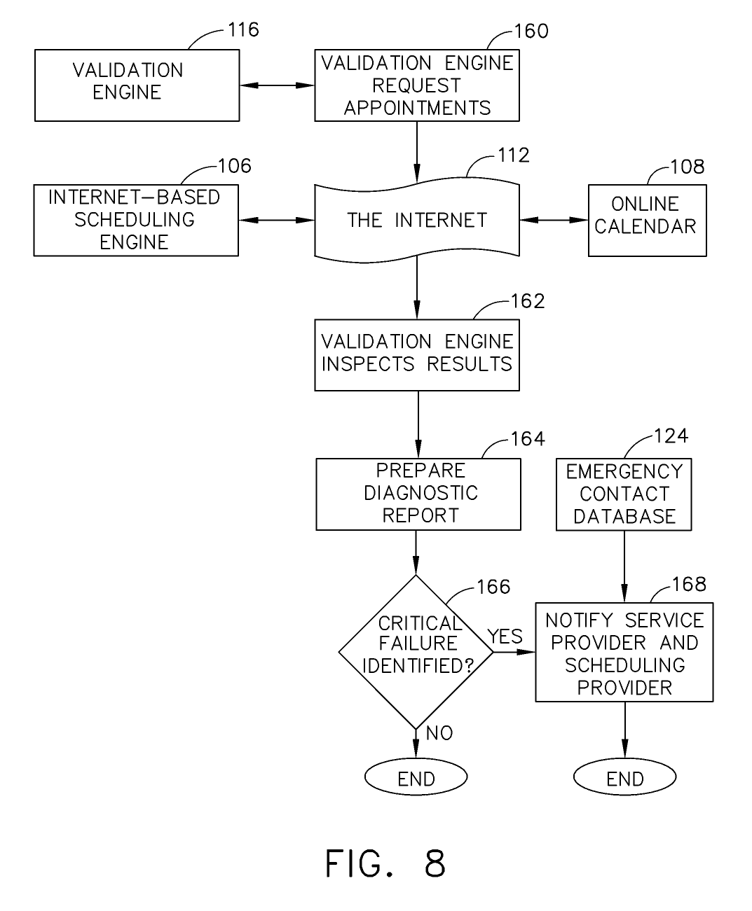

As shown in FIG. 8, the validation engine 116 may perform the web diagnostics. As shown at block 160, the validation engine 116 may access the provider's client interface, and may enter every type of appointment using a special code to designate a test customer. The internet-based calendar system may process the request. However, the request may be intercepted by the validation engine 116 rather than being forwarded to the provider. As shown at block 162, the validation engine 116 may compare the incoming request with the request that should have been received. Any failing websites or broken links may be identified. A diagnostic report may be prepared, as shown at block 164. As shown at block 166, if a failure that is critical to the provider's business is identified, then the validation engine 116 may contact the individuals in the emergency contact database 124 (FIG. 1) in the manner described above to provide notice of the failure, as shown at block 168.

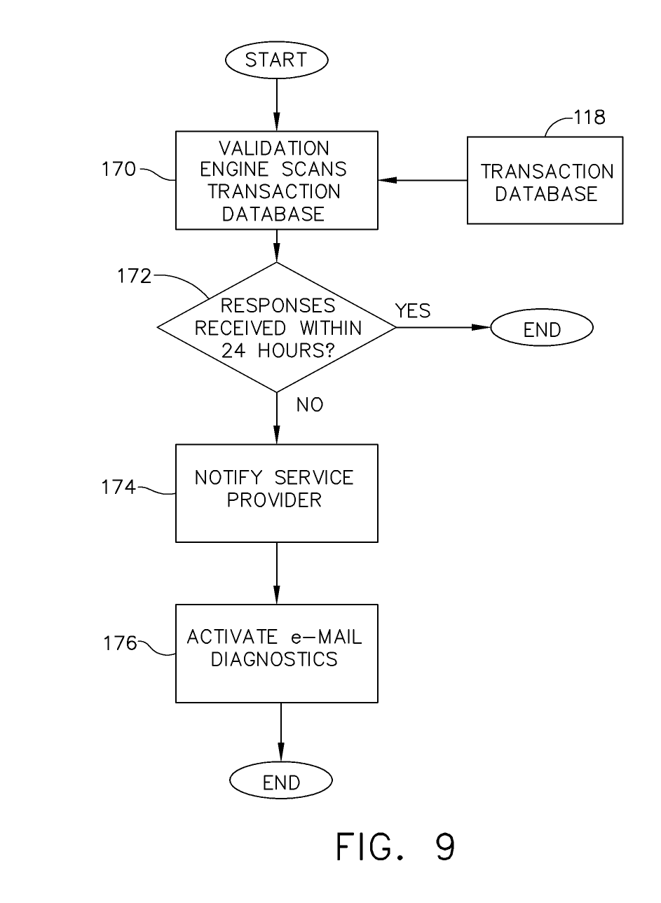

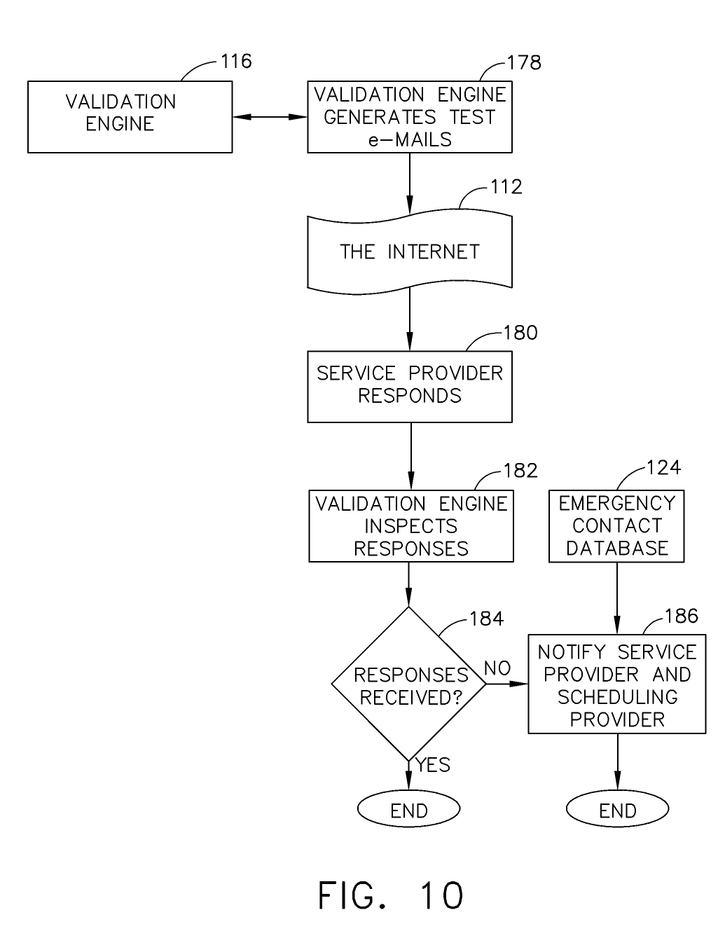

As noted above, the validation engine 116 (FIG. 1) may also assess the integrity of the provider (email) side of the Internet-based scheduling system 100. Referring to FIG. 9, in one particular implementation, beginning at block 170, the validation engine 116 may scan the transaction database 118 at regular intervals (e.g., every 10 minutes). Decision block 172 may query whether there have been any responses by the provider within a given time period (e.g., 24 hours). If not, then an exceptional transaction may be deemed to have occurred and a report may be sent to the provider (block 174) and the email diagnostics may be run by the validation engine (block 176).

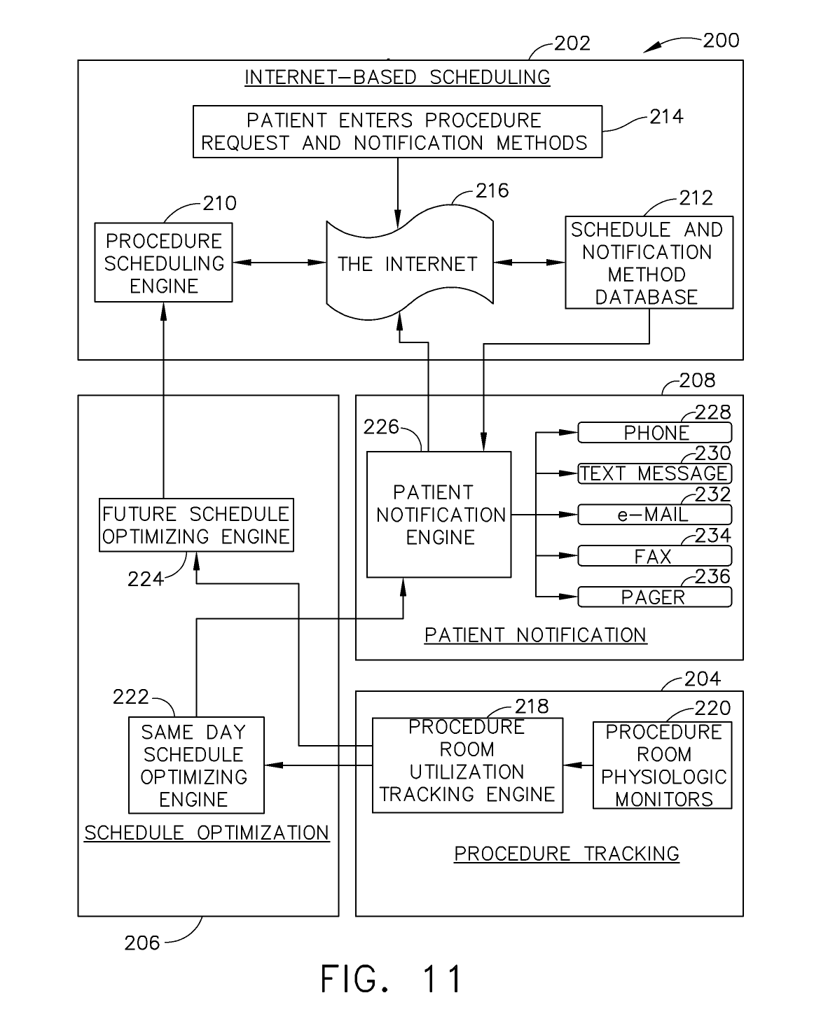

As shown in FIG. 10, the validation engine 116 may perform the email diagnostics. Specifically, at block 178, the validation engine 116 may generate test emails and may send them to the provider (block 180) over the Internet 112. The provider must respond with a given time period. At block 182, the validation engine 116 may inspect the response, if any, from the provider. Decision block 184 may query whether responses have been received. If the provider failed to respond, or the responses do not match the expected responses, then the validation engine 116 may contact the individuals in the emergency contact database 124 in the manner described above to provide notice of the failure, as shown at block 186.

Accordingly, the disclosed scheduling system 100 improves integrity and reliability of Internet scheduling by linking the Internet-based scheduling component 102 with the monitoring and validation component 104, thereby ensuring that all client requests for appointments receive a timely response, while also verifying the integrity of the client and provider components of the scheduling system and promptly identifying any failure of the client (web) or service provider (email) side of the scheduling system 100.

In a second aspect, the disclosed scheduling system may utilize clinical monitors to track the availability of medical procedure rooms and to automatically notify patients when to arrive, thereby potentially enhancing day-of-procedure scheduling between doctor (i.e., the provider) and patient (i.e., the client).

Pursuant to modern medical practice, patients are typically continuously electronically monitored during procedures. For example, even the most routine and non-invasive procedures include electronic monitoring of blood pressure and heart rate. Other typical physiological monitors include pulse oximetry (oxygen saturation) and electrocardiography. These are considered “standard of care” for sedation, and virtually all patients are monitored with pulse oximetry and electrocardiography from shortly after arriving in the procedure room until shortly before leaving the procedure room. Both pulse oximeters and electrocardiograms produce digital outputs of their waveforms that can be collected, analyzed and stored by a central computer, as occurs with Automated Anesthesia Record Systems. It has now been discovered that such monitors can be used to track the progress of a procedure by knowing when a room has an ongoing procedure and when the room is vacant. This information can be captured and used to improve future case scheduling, as well as optimal scheduling of procedure rooms on the day of the procedure.

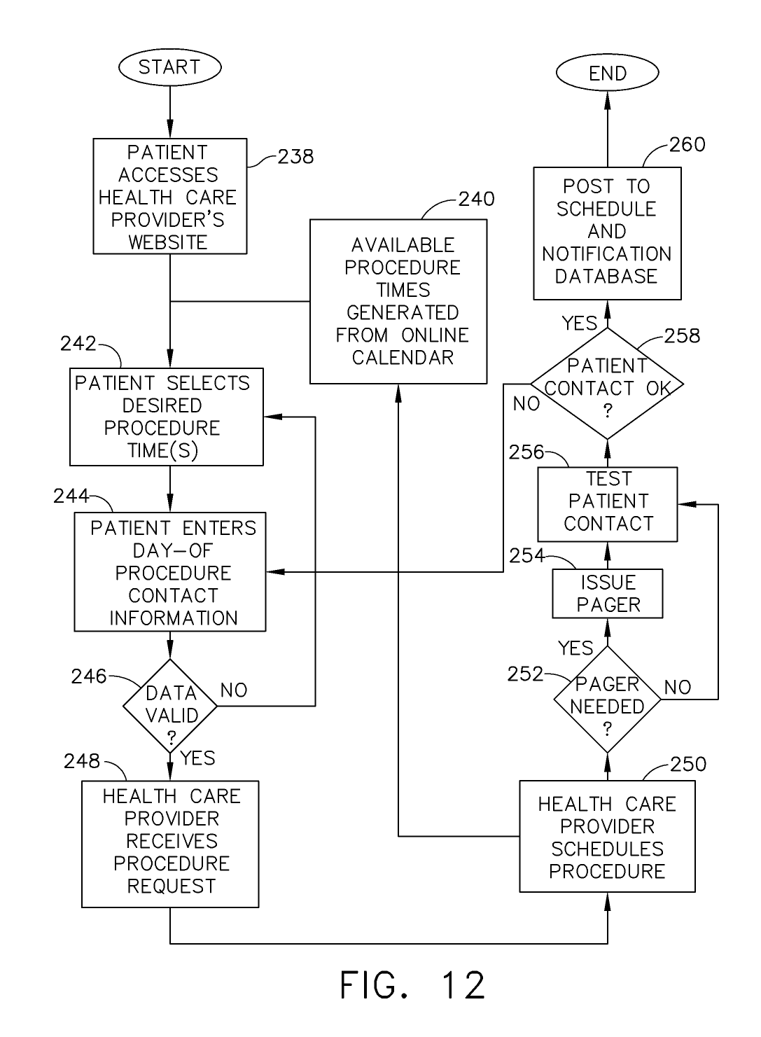

As shown in FIG. 11, a first embodiment of the second aspect of the disclosed scheduling system, generally designated 200, may include an Internet-based scheduling component 202, a procedure tracking component 204, a schedule optimization component 206 and a patient notification component 208. The components 202, 204, 206, 208 of the scheduling system 200 may cooperate to optimize day-of-procedure scheduling, as well as future case scheduling.

The Internet-based scheduling component 202 may include a procedure scheduling engine 210 and a schedule and notification method database 212. As shown at block 214, a patient may initiate a request for a procedure over the Internet 216. Once a request is initiated, the procedure scheduling engine 210 may access the schedule and notification method database 212, identify available appointment times, and communicate the available appointment times to the patient. The patient may select the desired appointment time (or multiple appointment times) and may indicate how the patient wishes to be contacted and notified about when to arrived to the medical facility on the day of the appointment.

Thus, the Internet-based scheduling component 202 may provide the patient with a platform for requesting a procedure. At this point, those skilled in the art will appreciate that the appointment time initially communicated to the patient may be a date, but not a time, or may be a date and an estimated time.

The procedure tracking component 204 may include a procedure room utilization tracking engine 218 and procedure room physiologic monitors 220. The procedure room physiologic monitors 220 may collect real-time data of patient vital signs in each procedure room and the procedure room utilization tracking engine 218 may analyze the data to determine the progress of procedures in various procedure rooms. Therefore, using various algorithms, the procedure room utilization tracking engine 218 may match each room to the daily schedule and may send the information to the schedule optimization component 206.

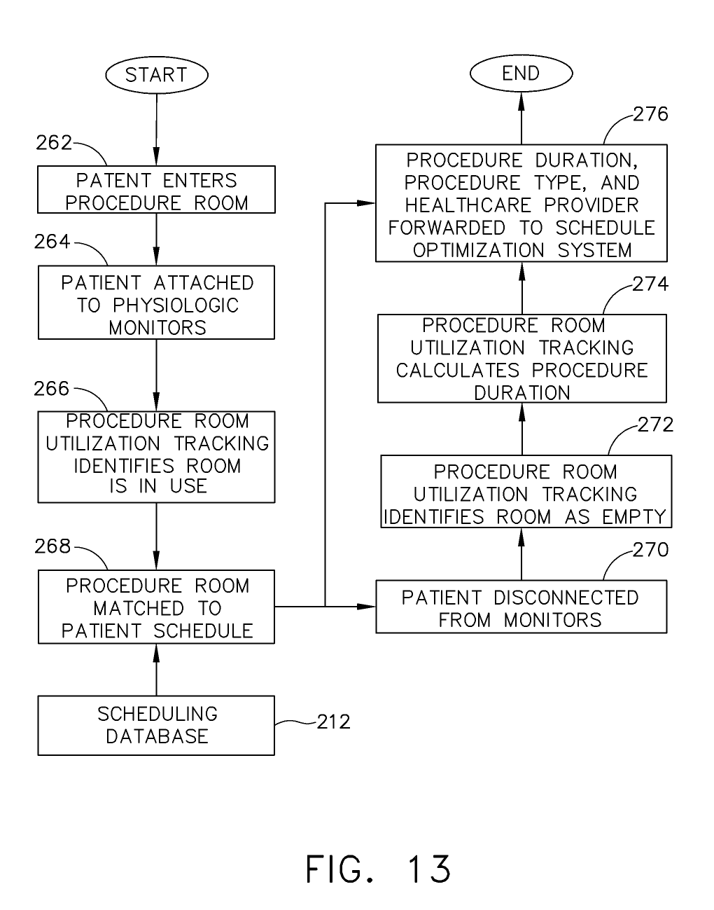

The procedure tracking component 204 may operate as shown in FIG. 13. Shortly after the patient enters the procedure room (block 262), physiologic monitors may be attached to the patient (block 264). As shown at block 266, the procedure room utilization tracking engine 218 (FIG. 11) may pick up the physiologic signals, and may identify whether a patient is in the room. The room may now be considered “in use.” Linking back to the schedule and notification method database 212 (FIG. 11), the procedure room utilization tracking engine 218 may match the room to the patient on the schedule, as shown at block 268. In a first implementation, this step may be performed entirely automatically. In a second implementation, human validation of the link may be requested. In a third implementation, the patient may be matched by name, age and medical record number to the name entered in the procedure room monitors. The information that the patient is in the room may be forwarded to the schedule optimization component 206 (FIG. 11).

The procedure room utilization tracking engine 218 may continue to read the patient vital signs to identify when the patient is disconnected from the monitors (block 270). Once the patient is disconnected, the procedure room utilization tracking engine 218 identifies the room as empty, as shown at block 272. Then, the procedure room utilization tracking engine 218 may calculate the procedure duration (block 274), which, together with the specific procedure and the specific health care provider, may be sent to the schedule optimization component 206 (FIG. 11), as shown at block 276.

The schedule optimization component 206 may include a same-day schedule optimizing engine 222 and a future schedule optimizing engine 224. The same-day schedule optimizing engine 222 may receive the real-time information from the procedure tracking component 204. Using published algorithms, the same-day schedule optimizing engine 222 may determine the ideal time for patients to arrive at the facility for their procedures and may send this information to the patient notification component 208. Those skilled in the art will appreciate that the “ideal time” selected by the same-day schedule optimizing engine 222 may attempt to balance the inconvenience to the patient associated with arriving too early with the economic cost associated with a patient arriving too late. The same-day schedule optimizing engine 222 may also examine the case order and room assignment to optimize procedure room utilization, which may be done based on published algorithms.

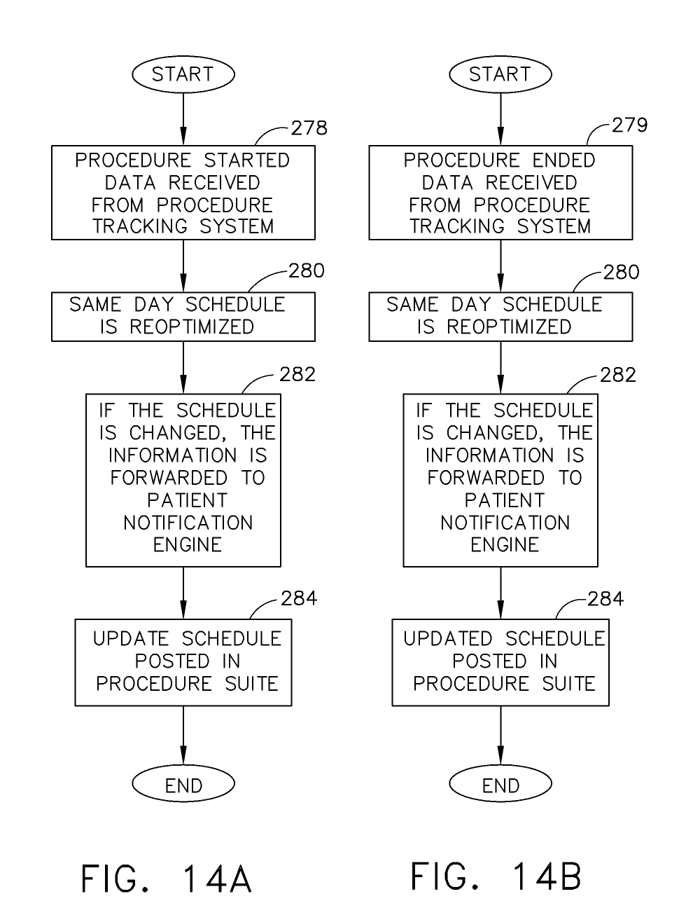

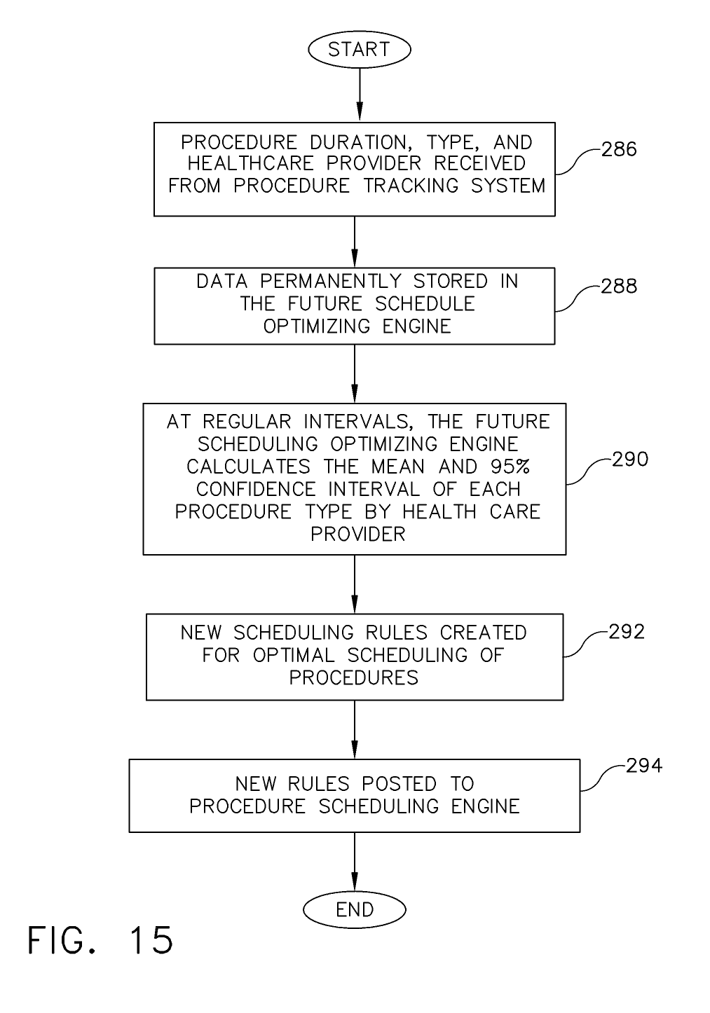

FIGS. 14A and 14B illustrate the interaction between the same-day schedule optimizing engine 222 and the procedure tracking component 204 to optimize the schedule on the day of the procedure. Specifically, FIG. 14A shows the arrival of information that a particular case has started (block 278), while FIG. 14B shows the arrival of information that a particular case has ended (block 279). In either case, the same-day schedule optimizing engine 222 analyzes the data to identify the optimum operating room schedule, and to identify optimal patient arrival times using published algorithms, as shown at block 280. At block 282, if the optimal patient arrival times have changed, then the new schedule is sent to the patient notification engine 226. Additionally, the updated schedule calculated by the same-day schedule optimizing engine 222 may be posted in the procedure suite, as shown at block 284, in the event that it may be necessary to place patients into different rooms.

The schedule optimization component 206 may also send the real-time information from the procedure tracking component 204 to the future schedule optimizing engine 224. The future schedule optimizing engine 224 may capture the individual procedure times and create a database of actual procedure times, which may be identified by procedure and/or service provider, among other things. The future schedule optimizing engine 224 may use this data to improve the scheduling of future patients by adjusting procedure time allotments based on historical data.

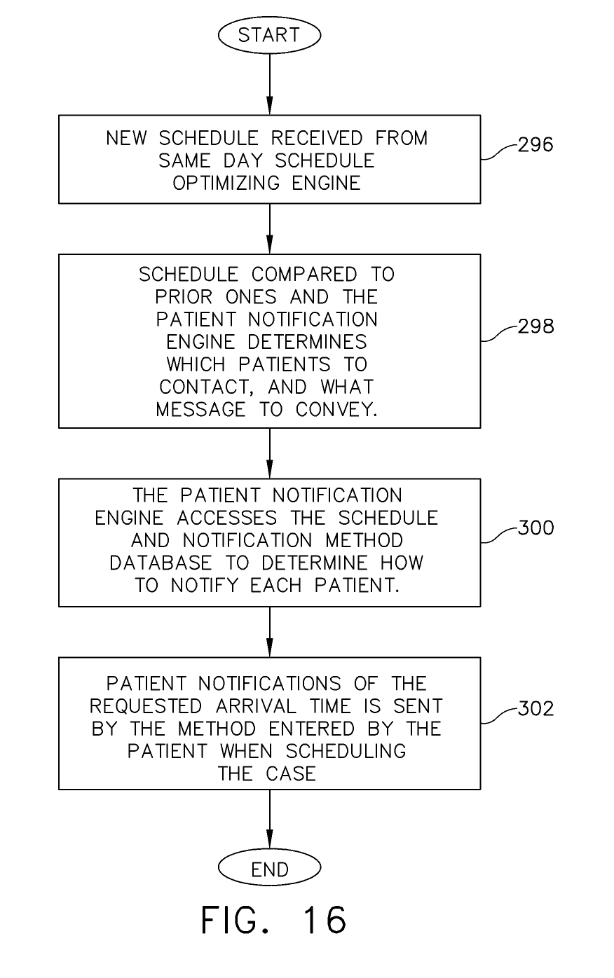

FIG. 15 illustrates the interaction between the future schedule optimizer engine 224 and the procedure tracking component 204. As shown at block 286, the future schedule optimizing engine 224 receives the procedure duration, procedure type, date, and health care provider identifier from the procedure tracking component 204. As shown at block 288, the data may be permanently stored in the future schedule optimizing engine 224. At regular intervals (e.g., monthly, quarterly, etc.), the future schedule optimizing engine 224 may access the database and may calculate statistics on the duration of each procedure for each health care provider, as shown at block 290. The calculations may be made using published algorithms. At block 292, the data may be used to create a new set of procedure scheduling rules for the procedure scheduling engine 210 and the new rules may be posted (block 294) to the procedure scheduling engine 210.

Thus, the schedule optimization component 206 may track service providers and procedure-room utilization in real time, and may constantly update the remaining daily schedule with predicted procedure start times. Furthermore, the schedule optimization component 206 may maintain and analyzes health care provider specific historical data on procedure times to guide future scheduling.

The patient notification component 208 may include a patient notification engine 226 that communicates with patients by, for example, telephone 228, text message 230, email 232, facsimile 234 and/or pager 236. Therefore, when the patient notification component 208 receives a message from the same-day schedule optimizing engine 22, it may send a notice to the patient based on the notification method requested by the patient in block 214. In one particular implementation, an alphanumeric pager may be issued to the patient by the health care provider when the procedure is scheduled, thereby ensuring a reliable means of communication on the day of the procedure.

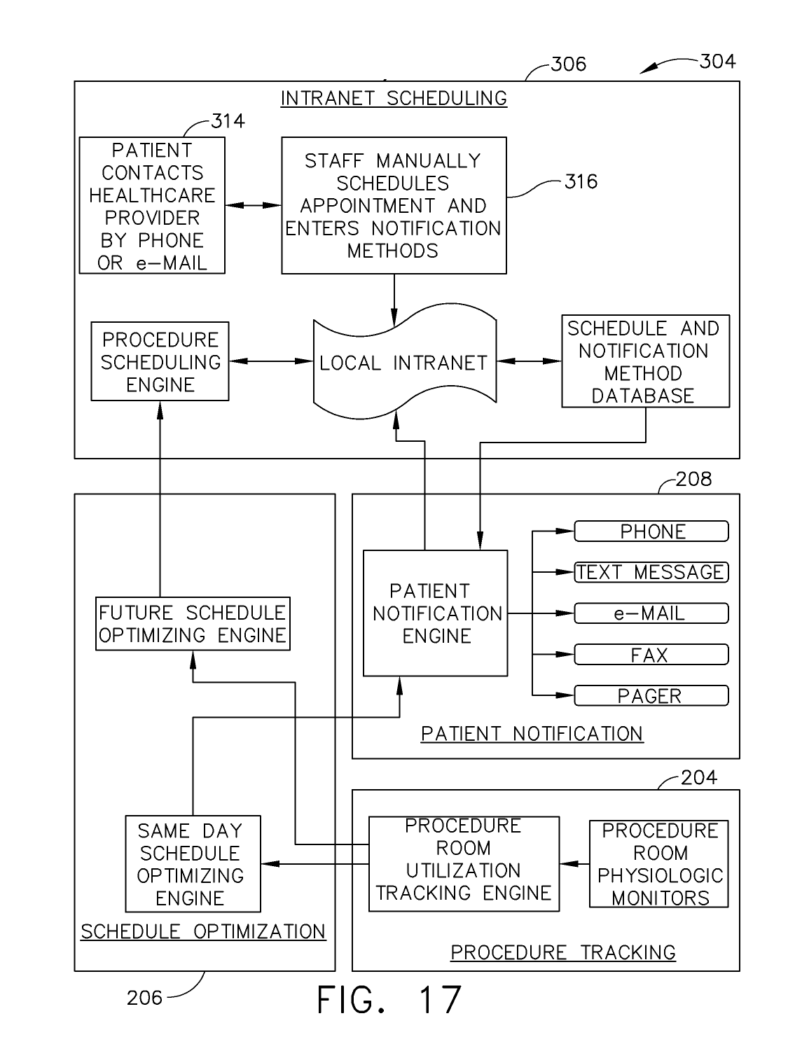

FIG. 16 illustrates the interaction between the same-day schedule optimizing engine 222 and the patient notification component 208. As shown at block 296, a new schedule may be received from the same-day schedule optimizing engine 222. At block 298, the patient notification engine 226 may examine the new start times, and may compare them with the start times previously transmitted to each patient (if any). The patient notification engine 226 may also consider the necessary lead time, and may make certain that every patient whose scheduled start time is within the requested lead time has received a notification. From these calculations, the system may develop a list of patients to be contacted and the contact message. As shown at block 300, the patient notification engine 226 may then communicate with the schedule and notification method database 212 to determine how to deliver the messages prior to actually delivering the messages, as shown at block 302.

FIG. 12 illustrates the logical flow of a patient using the scheduling system 200 to request a procedure. At block 238, the patient may access the health care provider's website and may request an appointment. As shown at block 240, the Internet-based scheduling component may access the health care provider's online procedure calendar and may identify a series of appointment times when the services could be provided to the patient. At block 242, the patient may select one or more suitable times for the procedure. For example, the patient may be asked to select three acceptable time slots, which provides for alternatives in case several patients concurrently select the same slot. Then, as shown at block 244, the patient may enter how he or she wishes to be contacted (e.g., telephone, text message, email, facsimile or alphanumeric pager). In one particular implementation, at least two methods need to be provided. The system 200 may also ask the patient how much lead time must be provided on the day of the procedure, to accommodate patients who may be a long distance away from the facility. At block 246, the data may be validated. For example, the contact information provided by the patient may be tested.

Once the data has been validated (block 246), the request may be forwarded to the health care provider (block 248) and the provider may schedule the procedure (block 250). If a pager is needed (block 252), arrangements may be made for delivery of the pager to the patient (block 254) and then the pager may be tested (block 256). The system may then test (block 258) the contact information given by the patient, requiring, for example, the patient to respond via the website. If the patient does not respond after several requests, the contact information may be reconfirmed. However, if the patient receives the messages and responds appropriately, then scheduling may be complete, and the appointment may be posted to the schedule and notification method database 212 (FIG. 11), as shown at block 260.

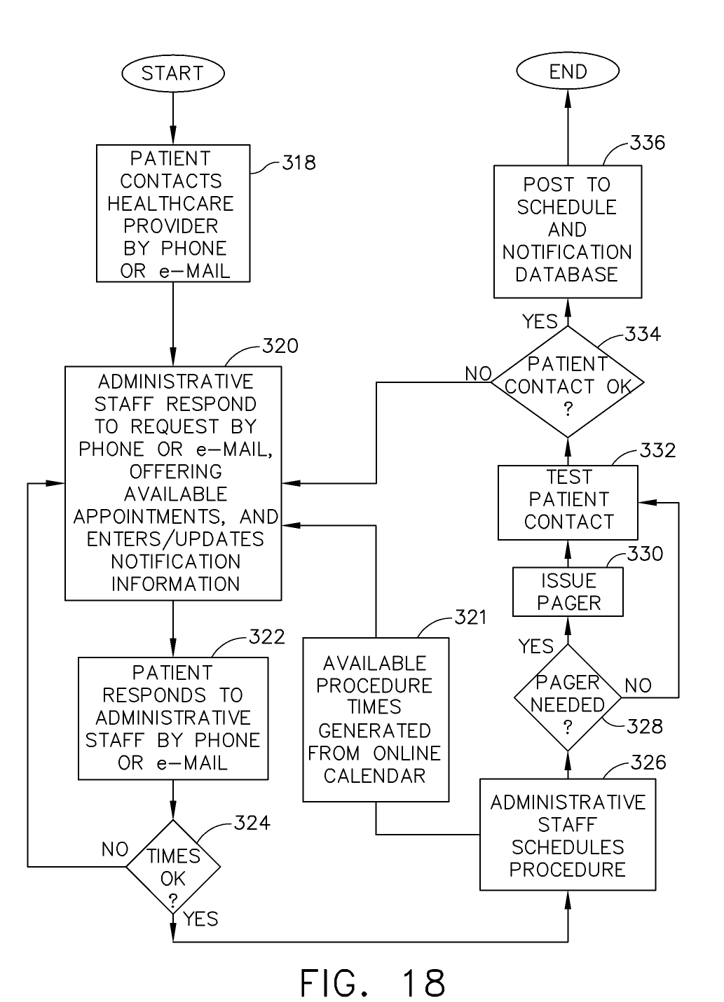

As shown in FIG. 17, a second embodiment of the second aspect of the disclosed scheduling system, generally designated 304, may include a local intranet-based scheduling component 306, a procedure tracking component 204, a schedule optimization component 206 and a patient notification component 208. The procedure tracking component 204, the schedule optimization component 206 and the patient notification component 208 may be the same as discussed above in connection with system 200.

As shown at block 314, the patient may communicate with the health care provider by contacting the administrative staff by phone or email and indicating a desire to schedule a procedure. In discussion by phone or email, a mutually suitable time may be determined. Once the patient has agreed to an available appointment time, the administrative staff may manually enter the schedule into the local intranet-based scheduling component 306, as shown at block 316, as well as specific information so the system can contact the patient prior to the procedure to provide the day-of-procedure schedule updating. The other features of system 304 may be the same as in system 200.

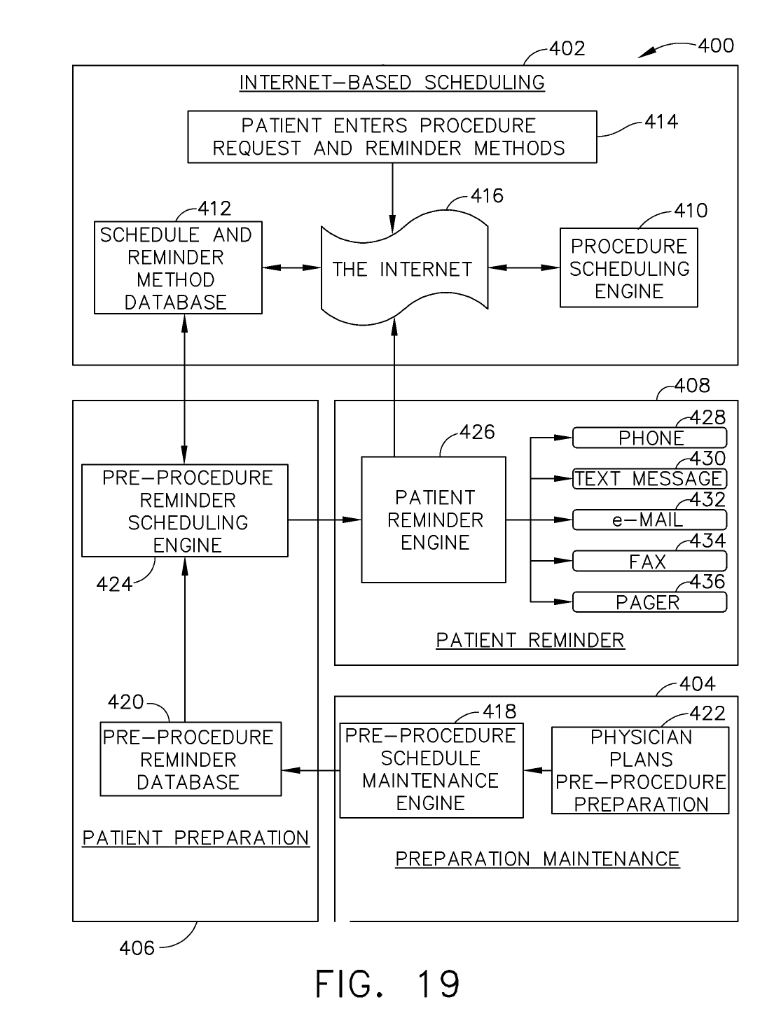

FIG. 18 illustrates the logical flow of a patient using the intranet-based scheduling system 304 to request a procedure. At block 318, the patient may contact the health care provider by phone or email. At blocks 320 and 321, the administrative staff may access the health care provider's calendar and, by phone or e-mail, offers the patient one or more available appointment times. At block 322, the patient selects an appointment time and provides contact information for day-of-procedure notification.

Once the data has been validated (block 324), the administrator may schedule the procedure (block 326). If a pager is needed (block 328), arrangements may be made for delivery of the pager to the patient (block 330) and then the pager (or other patient contact information) may be tested (block 332). At decision block 334, if the patient does not respond after several requests, the contact information may be reconfirmed. However, if the patient receives the messages and responds appropriately, then scheduling may be complete, and the appointment may be posted to the schedule and notification method database, as shown at block 336.

Accordingly, disclosed scheduling systems 200, 304 may increase same day procedure room efficiency, may decrease patient wanting times, and may capture procedure duration by heath care provider to optimize future scheduling. Systems 200, 304 may also provide significant economic benefits to health care providers, while increasing patient satisfaction with the services rendered.

In a third aspect, the disclosed scheduling system may provide timely reminders to clients about pre-appointment preparation instructions to increase compliance with those instructions. For example, in the medical profession, many procedures require extensive preparation on the part of the patient. In some cases, the requested procedure cannot be performed if the patient has not adequately followed the pre-appointment preparation instructions. As a specific example, inadequate bowel preparation is one of the most common causes of failed colonoscopy. Therefore, inadequate pre-appointment preparation can have significant economic impact on both the client (e.g., the patient) and the service provider (e.g., the medical doctor).

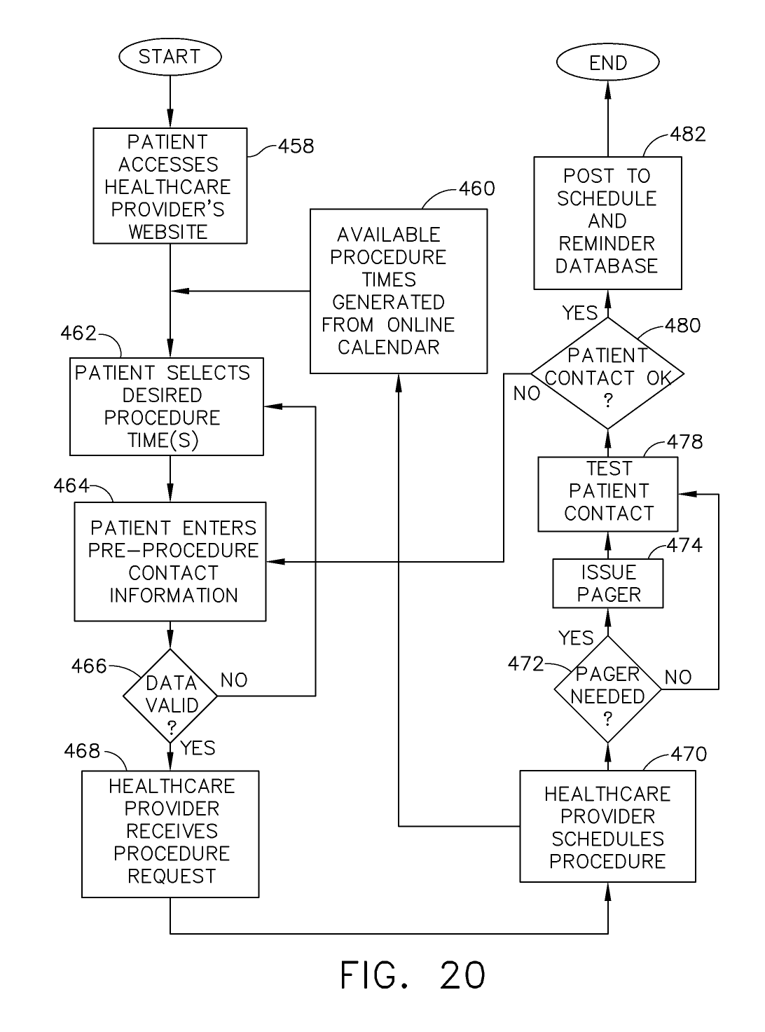

As shown in FIG. 19, one embodiment of the third aspect of the disclosed scheduling system, generally designated 400, may include an Internet-based scheduling component 402, a preparation maintenance component 404, a patient preparation component 406 and a patient reminder component 408. The components 402, 404, 406, 408 of the system 400 may cooperate as an integrated platform for improving client compliance with pre-appointment preparation instructions.

Examples of pre-appointment preparation instructions include (1) instructions (e.g., timing and dosage) for taking bowel preparation solution and (2) instructions to stop eating at midnight on the night before the procedure. Other pre-appointment preparation instructions will be left to the skilled artisan.

The Internet-based scheduling component 402 may include a procedure scheduling engine 410 and a schedule and reminder method database 412. As shown at block 414, a patient may initiate a request for a procedure over the Internet 416. Once a request is initiated, the procedure scheduling engine 410 may access the schedule and reminder method database 412, identify available appointment times, and communicate the available appointment times to the patient. The patient may select the desired appointment time (or multiple appointment times) and may indicate how the patient wishes to be contacted and reminded about pre-appointment preparation instructions.

As shown at block 422 of the preparation maintenance component 404, the provider may outline a schedule of preparations for each type of procedure to be performed. The outline may include the pre-appointment preparation instructions to be communicated to the patient, as well as an indication of when the instructions should be communicated to the patient relative to the scheduled appointment time. In one particular implementation, the preparation maintenance component 404 may include a pre-procedure schedule maintenance engine 418 that may allow the provider to generate and maintain the schedules, and which may store all the schedules in the pre-procedure remainder database 420 of the patient preparation component 406.

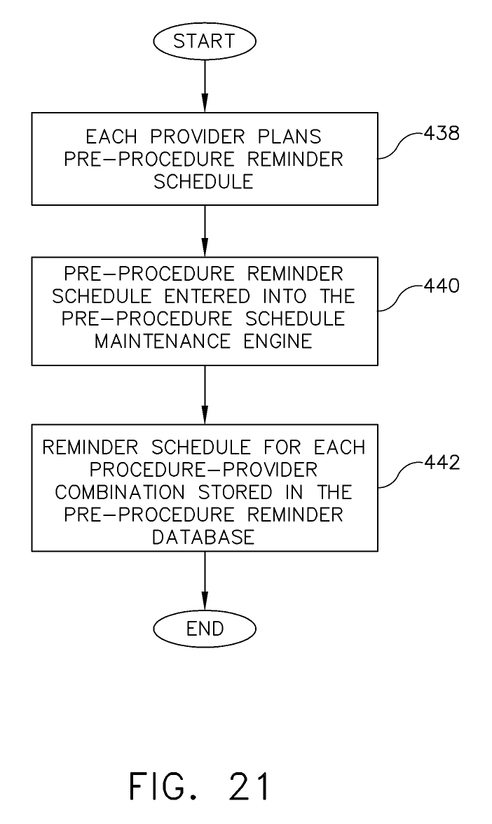

The preparation maintenance component 404 may be used as shown in FIG. 21. At block 438, each provider may develop the schedule of pre-appointment preparation instructions for each procedure performed by the provider. At block 440, the schedules may be entered into the pre-procedure schedule maintenance engine 418 (FIG. 19). For example, the pre-procedure schedule maintenance engine 418 may understand multiple time scales and may be capable of differentiating between preparations that occur a particular number of hours before a procedure (e.g., take an antibiotic 3 hours before the procedure) versus preparations that occur at a specified time of day prior to the procedure (e.g., nothing to eat or drink after midnight the night before the procedure). At block 442, the pre-procedure schedule maintenance engine 418 may save the schedule of patient preparations in the pre-procedure reminder database 420 (FIG. 19).

The patient preparation component 406 may include a pre-procedure reminder scheduling engine 424 and the pre-procedure reminder database 420. The pre-procedure reminder scheduling engine 424 may access the pre-procedure reminder database 420 to obtain the schedule of pre-appointment preparation instructions. The pre-procedure reminder scheduling engine 424 may then process the list of scheduled procedures in the schedule and reminder method database 412. Based on the scheduled day and time of each upcoming procedure, the pre-procedure reminder scheduling engine 424 may prepare a list of pre-appointment preparation instructions to be delivered to the patient by the patient reminder component 408.

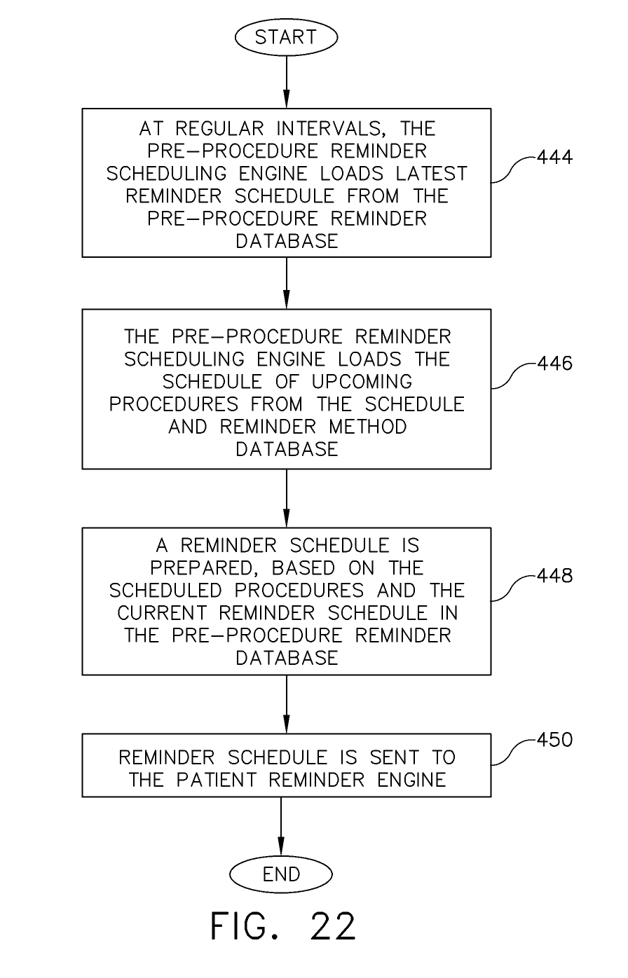

The patient preparation component 406 may be configured to perform the steps shown in FIG. 22. As shown at block 44, the pre-procedure reminder scheduling engine 424 may load the latest reminder schedule from the pre-procedure reminder database 420 at regular intervals. At block 446, the pre-procedure reminder scheduling engine 424 may also load the schedule of upcoming procedures from the schedule and reminder method database 412. At block 448, the pre-procedure reminder scheduling engine 424 may examine the upcoming scheduled procedures and may determine which pre-appointment preparation instructions should be sent at that time. At block 450, the current reminder schedule may be transmitted to the patient reminder engine 426.

The patient reminder component 408 may include a patient reminder engine 426 that may communicate the pre-appointment preparation instructions to the patient in an effort to achieve compliance with the instructions. The patient reminder engine 426 may communicate with the patient based on the reminder method selected by the patient, as discussed above. For example, the patient reminder engine 426 may communicate with the patient by telephone (block 428), text message (block 430), email (block 432), facsimile (block 434) and/or alphanumeric pager (block 436). In one particular implementation, an alphanumeric pager may be issued by the provider to the patient when the procedure is scheduled, thereby ensuring a reliable means for communicating the pre-appointment preparation instructions to the patient.

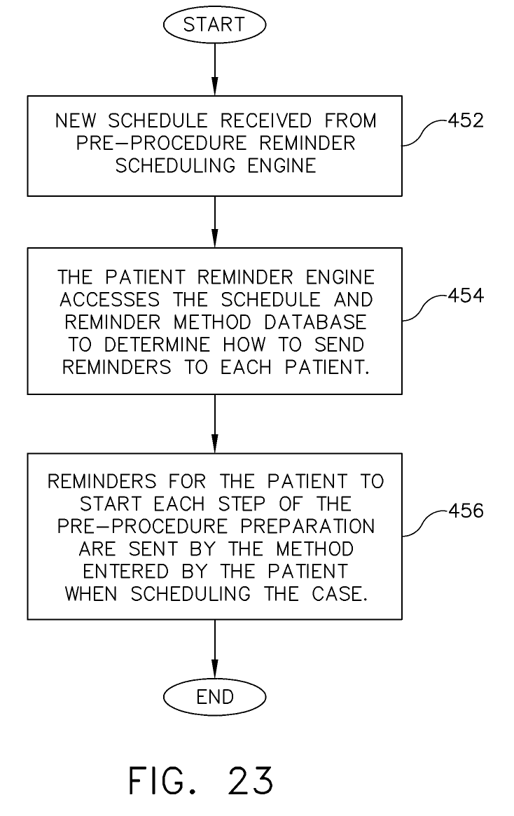

The pre-procedure reminder scheduling engine 424 may interact with the patient reminder component 408 as shown in FIG. 23. At block 452, the schedule of reminders may be received from the pre-procedure reminder scheduling engine 424. At block 454, the patient reminder engine 426 may access the schedule and reminder method database 412 to determine how the pre-appointment preparation instructions should be delivered. Then, at block 456, the instructions are delivered.

FIG. 20 illustrates the logical flow of a patient using the scheduling system 400 to request a procedure. At block 458, the patient may access health care provider's website and may request an appointment. As shown at block 460, the Internet-based scheduling component may access the health care provider's online procedure calendar and may identify one or more appointment times when the services could be provided to the patient. At block 462, the patient may select one or more suitable times for the procedure. For example, the patient may be asked to select three acceptable time slots, which provides for alternatives in case several patients concurrently select the same slot. Then, as shown at block 464, the patient may specify how he or she wishes to be contacted (e.g., telephone, text message, email, facsimile or alphanumeric pager). At block 466, the data may be validated (e.g., the contact information provided by the patient may be tested).

Once the data has been validated (block 466), the request may be forwarded to the health care provider (block 468) and the provider may schedule the procedure (block 470). If a pager is needed (block 472), arrangements may be made for delivery of the pager to the patient (block 474) and then the pager (or other contact method) may be tested (block 478) (e.g., the patient may be asked to respond to the test via the provider's website). At decision block 480, if the patient does not respond after several requests, the contact information may be reconfirmed. However, if the patient receives the message and responds appropriately, then the scheduling may be complete, and the appointment may posted to the schedule and reminder method database 412 (FIG. 19), as shown at block 482.

Accordingly, the disclosed scheduling system 400 may improve client compliance with pre-appointment preparation instructions, which may provide economic benefits to service providers and/or improve client satisfaction with the services rendered.

Although various aspects of scheduling systems have been shown and described, modifications may occur to those skilled in the art upon reading the specification. The present application includes such modifications and is limited only by the scope of the claims.

What is claimed is:

1. A method for scheduling an appointment between a client and a provider, said method comprising the steps of:
establishing a transaction database and at least one transaction monitoring rule;
receiving a request from said client to schedule an appointment with said provider;
providing said client with at least one available appointment time for said appointment;
receiving a selected appointment time from said client and logging said selected appointment time in said transaction database, wherein said selected appointment time is chosen from said at least one available appointment time;
communicating said selected appointment time to said provider;
determining whether said provider has accepted said selected appointment time;
when said provider has accepted said selected appointment time, communicating a confirmation of said selected appointment time to said client and logging said acceptance of said selected appointment time in said transaction database; and
applying said transaction monitoring rule to said transaction database to identify the presence of an exceptional transaction in said transaction database.

2. The method of claim 1 wherein said establishing step includes establishing at least two transaction monitoring rules.

3. The method of claim 1 wherein said transaction monitoring rule specifies a maximum time interval between said receiving-a-selected-appointment-time step and said communicating-a-confirmation step.

4. The method of claim 1 wherein said transaction monitoring rule specifies names of clients permitted to schedule appointments.

5. The method of claim 1 wherein said transaction monitoring rule specifies names of clients prohibited from scheduling appointments.

6. The method of claim 1 wherein said transaction monitoring rule specifies a maximum number of said requests per client within a pre-determined amount of time.

7. The method of claim 1 wherein said transaction monitoring rule specifies a minimum number of said requests within a pre-determine amount of time.

8. The method of claim 1 wherein said request from said client is received over the Internet.

9. The method of claim 1 wherein said selected appointment time is communicated to said provider by email.

10. The method of claim 1 wherein said step of determining whether said provider has accepted said selected appointment time includes accessing a calendar maintained by said provider.

11. The method of claim 1 wherein said confirmation of said selected appointment time is communicated to said client by email.

12. The method of claim 1 further comprising the step of generating a notification about said exceptional transaction.

13. The method of claim 12 further comprising the step of communicating said notification to said provider.

14. (canceled)

15. A method for scheduling an appointment between a client and a provider, said appointment requiring a room, said method comprising the steps of:
scheduling a date for said appointment;
tracking utilization of said room using an electronic monitoring device;
determining a time of said appointment based on said tracking step; and
notifying said client of said time.

16-17. (canceled)

18. The method of claim 15 wherein said scheduling step is performed over the Internet.

19. The method of claim 15 wherein said electronic monitoring device is a physiological monitoring device.

20. The method of claim 15 wherein said electronic monitoring device includes at least one of a blood pressure monitoring device, a heart rate monitoring device, a pulse oximetry device and a electrocardiogram device.

21. The method of claim 15 wherein said tracking step includes using two of said electronic monitoring devices.

22. The method of claim 15 wherein said electronic monitoring device provides real-time data.

23. The method of claim 15 wherein said client is notified of said time by at least one of a telephone call, a text message, an email, a facsimile and an alphanumeric pager notification.

24. The method of claim 15 wherein said client is notified of said time on said date.

25. The method of claim 15 further comprising the step of collecting historical data based on said tracking step, and determining said time based on said tracking step and said historical data.

26-27. (canceled)

28. A method for scheduling an appointment between a client and a provider comprising the steps of:
scheduling said appointment;
establishing a pre-appointment preparation instruction for said appointment;
determining when said pre-appointment preparation instruction should be sent to said client; and
after said determining step, communicating said pre-appointment preparation instruction to said client.

29-38. (canceled)
